(19) United States
(12) Patent Application Publication
(10) Pub. No.: US 2009/0203851 A1
(43) Pub. Date: Aug. 13, 2009

(54) Process for the Preparation of a Hydroxy-Aromatic Resin: Hydroxy-Aromatic Resin, and Modification Thereof

(75) Inventor: Rudolfus Antonius Theodorus Maria Benthem Van, Limbricht (NL)

Correspondence Address: NIXON & VANDERHYE, PC, 901 NORTH GLEBE ROAD, 11TH FLOOR, ARLINGTON, VA, 22203, US

(73) Assignee: DSM IP ASSETS B.V., Heerlen (NL)

(21) Appl. No.: 12/301,672

(22) PCT Filed: Jun. 1, 2007
(86) PCT No.: PCT/EP2007/004876

§ 371 Date: Mar. 26, 2009

(30) Foreign Application Priority Data
Jun. 2, 2006 (EP) 06011438.6

(51) Int. Cl. C08F 283/00; C08G 14/04; C07C 69/76; C08G 65/38

(52) U.S. Cl. 525/418; 528/155; 560/61; 528/206; 528/86

(57) ABSTRACT

The invention relates to a hydroxy-aromatic resin prepared by bringing together and letting react a hydroxy-aromatic compound of formula (I) and an alkanol hemiacetal compound of formula (II).

Formula (I) is:

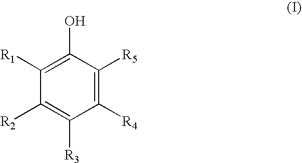

wherein R₁, R₂, R₃, R₄ and R₅ may be the same or may be different and are H, OH, a C₁-C₂₀ alkyl group, or an oligomeric or polymeric system, whereby at least one of the set consisting of R₁, R₃ and R₅ is H.

Formula (II) is:

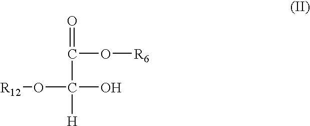

wherein R₈ is a C₁-C₁₂ alkyl group, aryl group, aralkyl group or cycloalkyl group and wherein R₁₂ is H, a C₁-C₁₂ alkyl groups aryl group, aralkyl group or cycloalkyl group.

The invention further relates to use of the resin in adhesives, laminates, and coatings.

The invention relates to a process for preparing a hydroxy-aromatic resin, to a hydroxy-aromatic resin, to a method for modifying a hydroxy-aromatic resin and to a resin such obtained.

Hydroxy-aromatic resins and their preparation are known, such as form example the preparation of phenol-formaldehyde resins from for example A. Knop, L. A. Pilato, Phenolic Resins, Springer Verlag Berlin 1990. These resins have many known uses, such as for example the use of these resins in adhesives for the preparation of particle boards.

A disadvantage of the known formaldehyde-containing hydroxy-aromatic resins is that their use is associated with health risks, relating to the emission of formaldehyde during resin preparation, resin curing and in end products.

It is the objective of the present invention to reduce or even eliminate the said disadvantage while still providing a compound suitable for the preparation of hydroxy-aromatic resins.

The said objective is achieved by a process for preparing a hydroxy-aromatic resin, comprising the steps of:
bringing together a hydroxy-aromatic compound of formula (I), an alkanol hemiacetal according to formula (II), optionally an amino compound, and optionally a catalyst to form a reaction mixture,
wherein:
   formula (I) is:

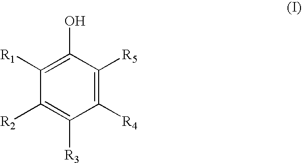

wherein R₁, R₂, R₃, R₄ and R₅ may be the same or may be different and are H, OH, a C₁-C₂₀ alkyl group, or an oligomeric or polymeric system, whereby at least one of the set consisting of R₁, R₃, and R₅ is H;
formula (II) is:

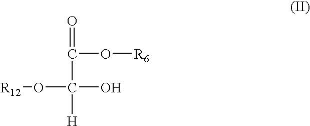

wherein R₆ is a C₁-C₁₂ alkyl group, aryl group, aralkyl group or cycloalkyl group and wherein R₁₂ is H, a C₁-C₁₂ alkyl group, aryl group, aralkyl group or cycloalkyl group;
bringing the reaction mixture to conditions whereby resin-forming takes place, whereby the hydroxy-aromatic resin is formed.

An advantage of the method according to the invention is that hydroxy-aromatic resins can be prepared that are essentially free of formaldehyde and thus suffer less, or even not at all, from the health risks associated with the use of formaldehyde, while still being suitable for use in typical known applications. Thus, resins prepared with the compound according to the present invention are in particular suitable for use in many applications such as adhesives, coatings, laminates, and shaped articles.

The method according to the invention relates to the preparation of a resin. A resin is herein understood to have the same meaning as it has to a skilled person in thermosetting chemistry, namely as a low molecular weight polymer having reactive groups. The term low molecular weight means a molecular weight typical for an oligomer and lying between a few hundred g/mole, e.g. 200, and a few thousand g/mole, e.g. 3,000. Ideally the number of reactive groups per molecule is at least two. These reactive groups form the chemical handles to connect the polymer chains together through covalent cross-link bonds, via a chemical reaction. The process of cross-linking is mostly referred to as “cure” or “hardening”. A resin may be present in the form of a solution, e.g. an aqueous solution, or as such.

The resin is according to the invention prepared by bringing raw materials together to form a reaction mixture. The raw materials comprise a hydroxy-aromatic compound according to formula (I). Hydroxy-aromatic compounds as such are known, and are defined as compounds having an aromatic ring with at least one —OH group attached directly to it. An example of such a compound is phenol. As is known in hydroxy-aromatic chemistry, the positions on the aromatic ring adjacent to and opposite the hydroxy group (i.e., ortho and para) have a different reactivity than the remaining two meta-positions. In formula (I), therefore, the groups R₁, R₃, and R₅ should be regarded within a similar context and are herein referred to as a set. In the hydroxy-aromatic compound, at least one of the groups in the set consisting of R₁, R₃, and R₅ is H; the other one or two groups in the said set—in case not all three of the said set is given by H—is/are OH, a C₁-C₂₀ or preferably a C₁-C₁₂ or C₁-C₉ alkyl group, or an oligomeric or polymeric system.

R₂ and R₄ may be the same or may be different and may each individually be H, OH, a C₁-C₂₀ or preferably a C₁-C₁₂ or C₁-C₉ alkyl group, or an oligomeric or polymeric system.

The oligomeric or polymeric system may be any suitable type such as hydroxy-aromatic resin, either of the resol or of the novolac type, preferably of the resol type; or it may be a different type of thermosetting or thermoplastic system.

The hydroxy-aromatic compound according to formula (I) may be one single compound but is understood to also comprise the meaning of a mixture of two or more compounds falling within the scope of the formulas as defined above. Examples of preferred compounds according to formula (IV) are phenol, (2, 3, or 4-)cresol, a meta-substituted phenol, resorcinol, catechol, (2, 3, or 4-)tert-butylphenol, (2, 3, or 4-)nonylphenol, (2,3-2,4-2,5-2,6- or 3,4-)dimethylphenol, (2, 3, or 4-)ethyl phenol, bisphenol A, bisphenol F, and hydrochinon. Further examples of preferred compounds according to formula (IV) are poly-phenolic systems such as tannins or lignins.

The raw materials that are brought together to form the reaction mixture comprise besides the hydroxy-aromatic compound as described above an alkanol hemiacetal according to formula (II). In formula (II), R₆ is a C₁-C₁₂ alkyl group, aryl group, aralkyl group or cycloalkyl group and R₁₂ is H, a C₁-C₁₂ alkyl group, aryl group, aralkyl group or cycloalkyl group. Preferably R₆ and R₁₂ are C₁-C₁₂ alkyl groups. Examples thereof are methyl, ethyl, propyl, butyl, pentyl, hexyl, and heptyl. R₆ and R₁₂ are in particular a methyl group or an ethyl group. The compound according to formula (II) may be one single compound but is understood to also comprise the meaning of a mixture of two or more compounds falling within the scope of the formulas as defined above. Examples of preferred compounds according to formula (II) are methylglyoxylate methanol hemiacetal (GMHA™, DSM Fine Chemicals, Linz); ethylglyoxylate ethanol hemiacetal (GEHA™, DSM Fine Chemicals, Linz); ethylglyoxylate methanol hemiacetal; butylglyoxylate butanol hemiacetal; butylglyoxylate methanol hemiacetal; butylglyoxylate ethanol hemiacetal; isopropylglyoxylate isopropanol hemiacetal; propylglyoxylate propanol hemiacetal; cyclohexylglyoxylate methanol hemiacetal and 2-ethylhexylglyoxylate methanol hemiacetal. Further examples of compounds according to formula (II) are glyoxylic acid hydrate, methylglyoxylate hydrate and ethylglyoxylate hydrate.

The raw materials that are brought together to form the reaction mixture may optionally comprise—besides the hydroxy-aromatic compound according to formula (I) and the alkanol hemiacetal according to formula (II) as described above—an amino compound. An amino compound is defined herein as a compound containing at least one —NH or —NH₂ group. Amino compounds are known as such; examples of amino compounds that are suitable for use in the method according to the invention are urea, melamine, melam and melem. Preferable, urea is used as amino compound.

The molar ratio between the raw materials that are brought together in the reaction mixture may vary between wide limits. The molar ration between the alkanol hemiacetal compound (A) and the hydroxy-aromatic compound (H), herein referred to as the A/H ratio, preferably lies between about 0.1 and about 10, more preferably between about 0.5 and about 3. If the reaction mixture also comprises an amino compound (O), then the ratios as given apply to the ratio between the alkanol hemiacetal compound and the sum of the hydroxy-aromatic compound and the amino compound. The molar ratio A/(H+O) is preferably at least 0.1, 0.2, 0.3, 0.4, 0.5 or 0.6 and preferably at most 10, 9, 8, 7, 6, 5, 4, 3, or 2.

If the molar A/H ratio or the molar A/(H+O) ratio lies above 1, resol-type of resins can be formed whereby reactive ‘A’-derived hydroxy groups are available. If the molar A/H ratio lies below 1, novolac-type of resins can be formed, in which essentially all ‘A’-derived hydroxy functionality has reacted away to form C—C and C—O ether bonds.

The bringing together of the raw materials to form the reaction mixture may be accomplished by simply mixing them; it may be beneficial to do this in the presence of a solvent. It may thus be beneficial to execute the reaction step according to the invention in a solvent or dispersant. As solvents, those compounds are suitable in which the reactants dissolve sufficiently to let the reaction take place. Examples of such solvents are water and various organic solvents. Depending on the specific compound or compounds of formula (I) and (II), it may well be possible to use one or more of the reactants as solvent; in such a case, it can be possible to forego on the use of a solvent that is essentially a non-reactant and to execute the reaction step in bulk. In particular, many of the compounds according to formula (II) are a liquid at temperatures between 10° C. and 100° C. and can act as dispersant/solvent as well as reactant.

Once the reaction mixture is formed, it should be brought to conditions whereby the hydroxy-aromatic resin can be formed, i.e. in a reaction step. Although the reaction step may proceed spontaneously once the respective compounds have been brought together, it may be useful to bring the compounds together in the presence of a catalyst in order to accelerate the reaction. As catalyst, preferably an acid is used; in particular, a Lewis or a Brønsted type of acid is preferred—such as for example sulphuric acid—whereby the pH is reduced to between 0 and 5, preferably to between 1 and 4, in particular to between 2 and 3. Suitable examples of acid catalysts are sulphuric acid, nitric acid, hydrochloric acid, phosphoric acid, boric acid, tetrafluoroboric acid, paratoluene sulphonic acid, methane sulphonic acid, formic acid, ammonium sulphate, ammonium chloride, ammonium nitrate, aluminum sulphate, aluminum chloride, zirconium (IV) chloride, titanium (IV) chloride, zinc chloride, stannic chloride, stannous chloride, boron trifluoride etherate.

The temperature in the reaction step of present process can vary within wide limits, and preferably lies between 10° C. and 100° C. More preferably the process is carried out at between 40° C. and 90° C. The pressure in the present process preferably is between 0.005 MPa and 1.0 MPa, preferably between 0.02 MPa and 0.2 MPa; most preferably, the pressure is atmospheric. The reaction step may be carried out in air, although it can have benefits to operate in an inert atmosphere such as nitrogen. The time needed for completion of the reaction step may vary within wide limits and is primarily determined by the time needed to achieve the end result of the reaction step, i.e. the formation of a resin. As is known, factors like the temperature and the nature and amount of catalyst strongly influence the time needed to achieve the desired end result. In practice, the reaction step could be completed in a time lying between 5 minutes and 180 minutes.

Depending on the nature of the raw materials, various intermediate structures have been identified within the framework of the present invention.

In one preferred embodiment of the invention, the hydroxy-aromatic compound of formula (I) is bisphenol-A, the alkanol hemiacetal of formula (II) is GMHA, and no amino compound is used. A primary adduct as formed in the reaction step was found to be the compound according to formula (III):

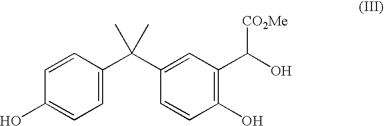

In formula (III) and in any other formulas as given in the context of the present invention, the indication Me refers to a methyl group. Further adducts as are typically formed in the resin-forming according to this embodiment of to the invention are given in formulas (IV), (V), (VI and (VII), to which the invention thus also relates:

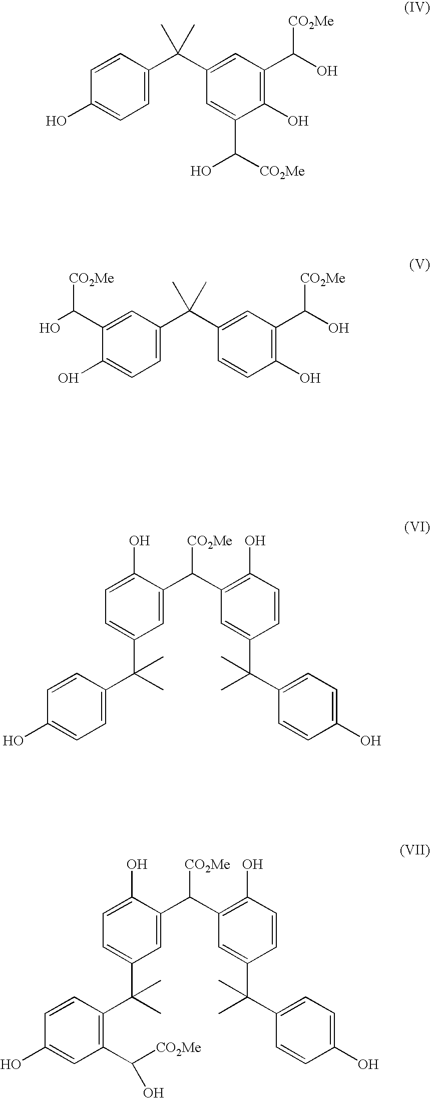

In another preferred embodiment of the method according to the invention, the hydroxy-aromatic compound of formula (I) is phenol, the alkanol hemiacetal of formula (II) is GMHA, and urea is chosen as amino compound. A primary adduct as formed in the reaction step was found to be the compound according to formula (VIII):

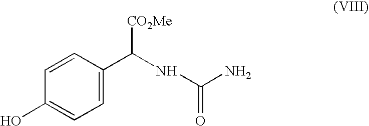

Further adducts as are typically formed in the resin-forming according to this embodiment of to the invention are given in formulas (IX) and (X), to which the invention thus also relates:

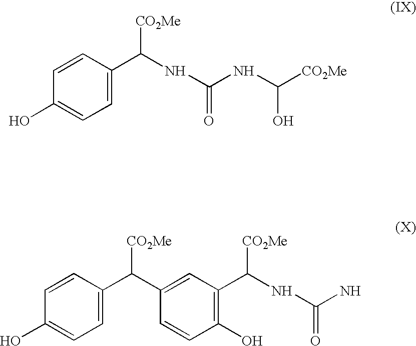

In a further embodiment of the method according to the invention, the hydroxy-aromatic compound of formula (I) is phenol, the alkanol hemiacetal of formula (II) is GMHA, and no amino compound is chosen. A primary adduct as formed in the reaction step was found to be the compound according to formula (XI):

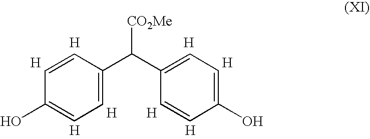

Further adducts as are typically formed in the resin-forming according to this embodiment of to the invention are given in formulas (XII), (XIII), (XIV), (XV), (XVI) and (XVII), to which the invention thus also relates. The preference for phenol to react on the para location or—somewhat less preferred—on the ortho location was confirmed.

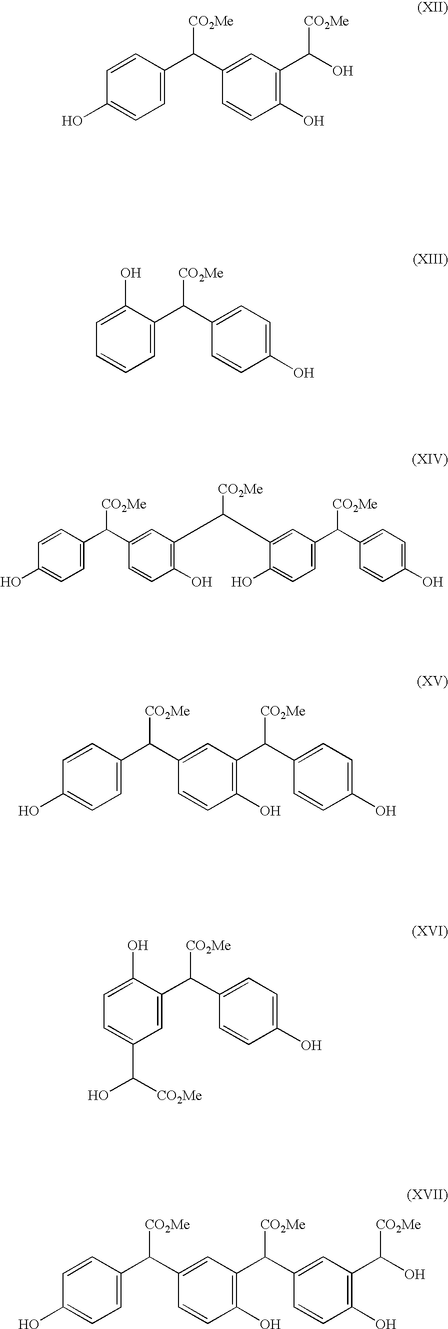

The invention further relates to the resin as obtainable by the method as described above. The invention moreover relates to the use of the hydroxy-aromatic aldehyde resin according to the invention for the preparation of coatings or shaped articles such as wood-based panels like particle boards and laminates, or mineral wool such as stone wool or glass wool. To this end, the resins may be used by methods and under conditions similar to those known per se from the use of known hydroxy-aromatic aldehyde resins like phenol-formaldehyde resins. A catalyst and other additives may be added to the resin before the resin is used for processing in its final application. Examples of customary additives are mould release agents, antistatic agents, adhesion promoters, plasticizers, colour enhancing agents, flame retardants, fillers, flow promoters, colorants, diluents, polymerization initiators, UV-stabilizers and heat stabilizers. Examples of fillers are glass fibres, mica, carbon fibres, metal fibres, clay, aramide fibres and strong polyethylene fibres.

It was found that if phenol was used as hydroxy-aromatic compound of formula (I), the amount of free phenol in the resin as prepared can be very low. This is surprising, since known phenolic resins such as phenol-formaldehyde resins are notorious for suffering from high levels of free phenol, ranging often in levels of around 1% or more. In the resin according to the invention, by contrast, the level of free phenol was found to be very low, often below 0.1% or even below 0.01%.

The resin according to the invention may be used as such; however, it is also possible to subject the resin to a modification step; this is a reaction step designed to alter or enhance its functionality in a specific way. An example of an altered functionality is the solubility of the resin in water. An example of an enhanced functionality is the addition of a reactive group. An example of a modification step is to bring the resin in contact with compounds that react with the —OH groups; an example of such a compound is epichlorohydrin. Another example of a modification step is to bring the resin in contact with compounds that react with the —OR₆ groups; an example of such a compound is water; the hydrolysis of the —OR₆ group into a —COOH group increases the solubility of the resin in water. Also, the modification step may be achieved through a transesterification reaction between the —OR₆ groups and suitable compounds such as amines; examples of amines are ethanolamine and diethanolamine (DEA). If a modification step with an amine is done on a resin, it is preferred that no amino compound was used as raw material for resin preparation.

In a preferred embodiment of the invention, the bisphenol compound of formula (XI) is used in the preparation of an epoxy resin. An epoxy resin, as is known, is an oligomeric or polymeric material comprising at least two oxygen-containing three-membered ring structures, often in the form of glycidyl ether moieties. The oxygen-containing three-membered ring serves as location for further reactions, commonly referred to as curing or cross-linking. The term epoxy resins is in practice also used for the cured/cross-linked polymers, even thought practically all or even all of the oxygen-containing three-membered ring structures that were present have reacted away. It is well-known that certain bisphenol compounds such as bisphenol A can be used to prepare epoxy resins, e.g. through the reaction with epichlorohydrin in the presence of NaOH. It was now found that bisphenol A may be partly or even wholly replaced by the bisphenol compound of formula (XI) to prepare epoxy resins. The invention thus relates to the use of the bisphenol compound of formula (XI) in epoxy resins, and to epoxy resins thus obtainable. Compared to the use of bisphenol A in epoxy resins, the epoxy resins according to the invention provide, due to the ‘—COOMe’ group as derived from the compound of formula (XI) additional possibilities for subsequent chemical modification or, due to the additional polarity of the said ‘—COOMe’ group, additional possibilities for adhesion of the resin to other materials. Moreover, the presence of the ‘—COOMe’ group enables the possibility that the compound of formula (XI) acts as branching agent. The invention thus further relates to the use of such epoxy resins in coatings, inks, structural composites, flooring, electrical laminates, or adhesives.

In another preferred embodiment of the invention, the hydroxy-aromatic resin is subjected to a modification step in which the resin is brought into contact with ammonia. The ammonia may be as such, e.g. in gaseous form or in liquid form, or it may be in the form of a solution, e.g. an aqueous solution. An important effect of the ammonia treatment is typically the increase in solubility of the resin in aqueous systems. Moreover, this increase in solubility has essentially no or only a limited effect on the ability of the resin to undergo subsequent curing reactions. It was found that certain other methods that may lead to increase of solubility of the resin in water, such as treatment with basic aqueous solutions of alkaline metals such as aqueous NaOH, can lead to a severe or even complete destruction of the ability of the resin to undergo curing reactions which is undesirable.

In yet another preferred embodiment of the invention, the hydroxy-aromatic resin is used in the preparation of thermoplastic polymers. In particular, compounds of formula (III), IV), (V), (VI), (VII), (VIII), (IX), (X), (XI), (XII), (XIII), (XIV), (XV), (XVI) or (XVII) can be used as monomer to replace part or all of known monomers such bisphenol A or other diols, or (aromatic) esters in processes for the preparation of polycarbonates or polyurethanes. It is noted hereby that compared to di-hydroxy-aromatic compounds such as bisphenol A, the compounds according to the invention comprise at least one aliphatic (none-aromatic) hydroxy group; a consequence thereof is that the incorporation of the compounds according to the invention into polymeric structures is easier, as aliphatic hydroxy groups can be better made to react than aromatic hydroxy groups.

In a further preferred embodiment of the invention, a compound of formula (III), IV), (V), (VI), (VII), (VIII), (IX), (X), (XI), (XII), (XIII), (XIV), (XV), (XVI) or (XVII) is, prior to being used as a monomer in the preparation of a polymeric material, subjected to a modification step. Preferred examples of such modification steps are ethoxylation or propoxylation.

The processes for the preparation of polyurethanes or polycarbonates as referred to are as such known; optimal conditions for incorporating the compounds according to the invention may be found through routine experimentation. The invention further relates to polyurethanes or polycarbonates thus obtainable.

The invention will be elucidated by means of the following examples, without being limited to it.

### EXAMPLE 1

A hydroxy-aromatic resin was prepared in the following fashion: as hydroxy-aromatic compound, 58.84 grams of bisphenol-A (97% purity) was taken; as alkanol hemiacetal, 66.73 grams of GMHA (90% purity) was taken. These components were mixed together, i.e. the bisphenol A was dissolved into the GMHA, at a temperature of 80° C. No further solvent was used. As catalyst, 0.5 ml of concentrated H₂SO₄ was added; the temperature was then raised to 90° C., and the reaction continued for 3 hours under nitrogen atmosphere and at reflux. Upon cooling, a very high viscosity resin was obtained that did not dissolve in water.

As subsequent treatment, a portion of the resin was taken and treated at 200° C. during 2 hours. This resulted in the formation of a glassy material, indicative of a cured resin. The glassy material contained less than 1 wt. % of either of the raw materials bisphenol A or GMHA in their free, unreacted form. Of this glassy material, 5 grams were taken and combined with 95 grams of demineralised water; then, the whole was heated to 80° C. during 3 hours. After cooling down and filtering, less than 1 wt. % of the 5 grams was lost due to degradation and dissolving.

Another portion of the resin was taken, and combined in 5 wt. % with demineralised water. Initially, no solution was formed. However, after the addition of ammonia in the form of an aqueous NH₄OH solution at 60° C. it turned out to be possible to dissolve the resin; at that moment the pH of the aqueous resin solution was 7.5. Subsequent to the ammonia treatment, the resin solution was heated to—ultimately—200° C.; this yielded—after evaporation of water and ammonia—a glassy material. This glassy material turned out to be as insoluble in water at 80° C. as prior to the ammonia treatment. This shows that although an ammonia treatment according to the invention served to create water-solubility of a resin, it did not lead to destruction of the resin as evidenced by the fact that it could be cured.

### EXAMPLE 2

A hydroxy-aromatic resin was prepared in the following fashion: as hydroxy-aromatic compound, 26.14 grams of a 90% solution of phenol in water was taken; as alkanol hemiacetal, 166.82 grams of GMHA (90% purity) was taken. As amino compound, 15.02 grams of urea was taken. Furthermore, 20.21 grams of demineralised water was used as solvent. These components were mixed together, i.e. the urea was dissolved into the GMHA/water/phenol mixture. As catalyst, 2.5 ml of concentrated H₂SO₄ was added; the temperature was then raised to 95° C., and the reaction continued for 6 hours under nitrogen atmosphere and at reflux. Upon cooling, a very high viscosity resin was obtained that did not dissolve in water.

As subsequent treatment, a portion of the resin was taken and treated at 200° C. during 2 hours. This resulted in the formation of a glassy material, indicative of a cured resin. Of this glassy material, 5 grams were taken and combined with 95 grams of demineralised water; then, the whole was heated to 80° C. during 3 hours. After cooling down and filtering, about 21 wt. % of the 5 grams was lost due to degradation and dissolving.

From this example, it is concluded that a hydroxy-aromatic resin according to the invention that also comprises an amino compound can be prepared. Without committing to any theory, it is thought that the urea as incorporated into the resin may have the effect of sensitizing the resin towards hydrolysis attack. A similar effect is known in phenol-urea-formaldehyde and melamine-urea-formaldehyde resins.

1. Process for preparing a hydroxy-aromatic resin, comprising the steps of:
bringing together a hydroxy-aromatic compound of formula (I), an alkanol hemiacetal according to formula (II), optionally an amino compound, and optionally a catalyst to form a reaction mixture,
wherein:
formula (I) is: 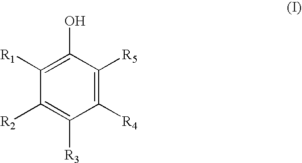
wherein R₁, R₂, R₃, R₄ and R₅ may be the same or may be different and are H, OH, a C₁-C₂₀ alkyl group, or an oligomeric or polymeric system, whereby at least one of the set consisting of R₁, R₃, and R₅ is H;
formula (II) is: 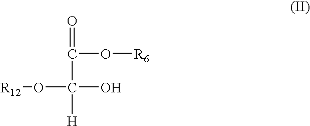
wherein R₆ is a C₁-C₁₂ alkyl group, aryl group, aralkyl group or cycloalkyl group and wherein R₁₂ is H, a C₁-C₁₂ alkyl group, aryl group, aralkyl group or cycloalkyl group;
bringing the reaction mixture to conditions whereby resin-forming takes place, whereby the hydroxy-aromatic resin is formed.

2. Process according to claim 1, wherein the hydroxy-aromatic compound of formula (I) is bisphenol-A.

3. Process according to claim 1, wherein the hydroxy-aromatic compound of formula (I) is a meta-substituted compound.

4. Process according to claim 3, wherein the hydroxy-aromatic compound of formula (I) is resorcinol.

5. Process according to claim 1, wherein an amino compound is brought together with the hydroxy-aromatic compound according to formula (I) and the alkanol hemiacetal according to formula (II) to form the reaction mixture, said amino compound selected from the group consisting of urea and melamine.

6. Process according to claim 1, wherein the alkanol hemiacetal according to formula (II) is chosen from the group consisting of methylglyoxylate methanol hemiacetal (GMHA™), ethylglyoxylate ethanol hemiacetal (GEHA™), ethylglyoxylate methanol hemiacetal, butylglyoxylate butanol hemiacetal, butylglyoxylate methanol hemiacetal, butylglyoxylate ethanol hemiacetal, isopropylglyoxylate isopropanol hemiacetal, propylglyoxylate propanol hemiacetal, cyclohexylglyoxylate methanol hemiacetal and 2-ethylhexylglyoxylate methanol hemiacetal.

7. Hydroxy-aromatic resin, obtainable by the process of claim 1.

8. Process for modifying a hydroxy-aromatic resin, wherein a resin according to claim 7 is brought into contact with a compound selected from the group consisting of alkylamines, dialkylamines and epichlorohydrin.

9. Bisphenol compound of formula (XI): 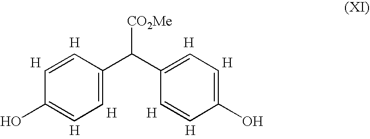
wherein Me is methyl.

10. Method of preparing epoxy resins, wherein the bisphenol compound of formula (XI) is used.

11. Epoxy resin, obtainable by the method of claim 10.

12. Use of an epoxy resin according to claim 11 in coatings, inks, structural composites, flooring, electrical laminates, or adhesives.

13. Process for the preparation of a polycarbonate, wherein at least one of the compounds of formula (III), IV), (V), (VI), (VII), (VIII), (IX), (X), (XI), (XII), (XIII), (XIV), (XV), (XVI) or (XVII) is used as a monomer or co-monomer.

14. Process for the preparation of a polyurethane, wherein at least one of the compounds of formula (III), IV), (V), (VI), (VII), (VIII), (IX), (X), (XI), (XII), (XIII), (XIV), (XV), (XVI) or (XVII) is used as a monomer or co-monomer.

15. Process for modifying a hydroxy-aromatic resin, wherein a resin according to claim 7 is brought into contact with ammonia.

16. Process according to claim 15, said ammonia being in gaseous or liquid form or in the form of an aqueous solution.

17. Hydroxy-aromatic resin, obtainable by the process of claim 15.

18. Use of a hydroxy-aromatic resin according to claim 7 in adhesive compositions.
